(19) United States
(12) Patent Application Publication
(10) Pub. No.: US 2014/0209096 A1
(43) Pub. Date: Jul. 31, 2014

(54) Portable Inhaler

(71) Applicant: Shaahin Cheyene, Venice, CA (US)

(72) Inventor: Shaahin Cheyene, Venice, CA (US)

(21) Appl. No.: 13/750,979

(22) Filed: Jan. 25, 2013

(51) Int. Cl. A61M 15/00

(52) U.S. Cl. CPC A61M 15/00
USPC 128/203.12

(57) ABSTRACT

The device is a portable inhalers. It is an electronic device that uses air pressure to deliver, flavor, aroma and possibly active elements utilizing air pressure, airflow and room temperature volatilization or vaporization. It has a ducted blower to blow room temperature air through a filter embedded with liquid, powder, paste or other source material. The user would insert the nozzle into the nose or mouth and inhale to enjoy the aroma and to receive the benefits of active elements.

### CROSS-REFERENCES TO RELATED APPLICATIONS (IF ANY)

None.

### BACKGROUND OF INVENTION

1. Field of the Invention

This device refers to the field of inhalers more practically an electronic device that uses air pressure to deliver, flavor, aroma and possibly active elements utilizing air pressure, airflow and room temperature volatilization or vaporization.

2. Background

Most current inhalers are larger and harder to use. They are bulky and use flame or a heating element to convert the active elements. This can lead to burns while the size makes them hard to use.

There is still room for improvement in the art.

### SUMMARY OF THE INVENTION

This device refers to the field of inhalers more practically an electronic device that uses air pressure to deliver, flavor, aroma and possibly active elements utilizing air pressure, airflow and room temperature volatilization or vaporization. The inhaler consists of a body with a nozzle. The inhaler uses a ducted blower to blow room temperature air through a filter embedded with liquid, powder, paste or other source material.

### BRIEF DESCRIPTION OF THE DRAWINGS

Without restricting the full scope of this invention, the preferred form of this invention is illustrated in the following drawings:

FIG. 1a is a top view of the device;

FIG. 1b is a bottom view of the device;

FIG. 2a is a side view of the device;

FIG. 2b is the other side view of the device;

FIG. 3a is a front view of the device;

FIG. 3b is a back view of the device;

FIG. 4 shows the air flow through the device;

FIG. 5 shows the components of the device; and

FIG. 6 shows a disposable nozzle.

### BRIEF DESCRIPTION OF THE PREFERRED EMBODIMENTS

There are a number of significant design features and improvements incorporated within the invention.

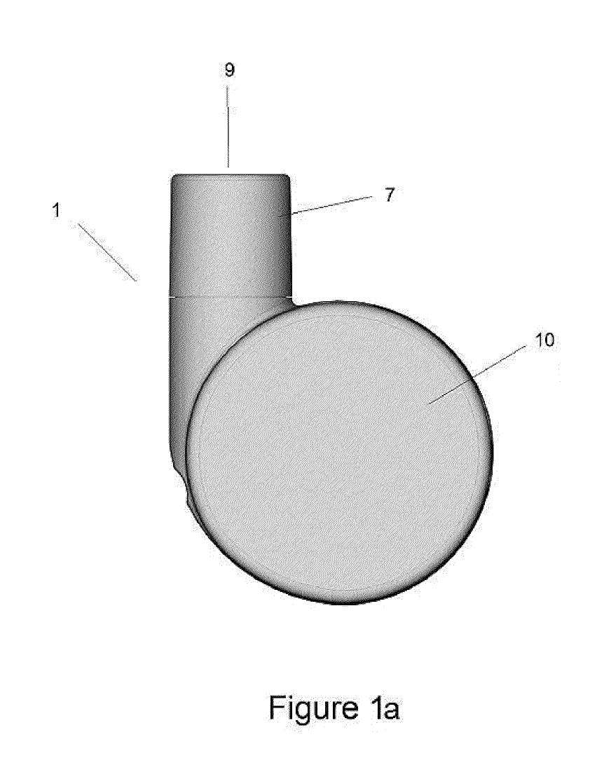

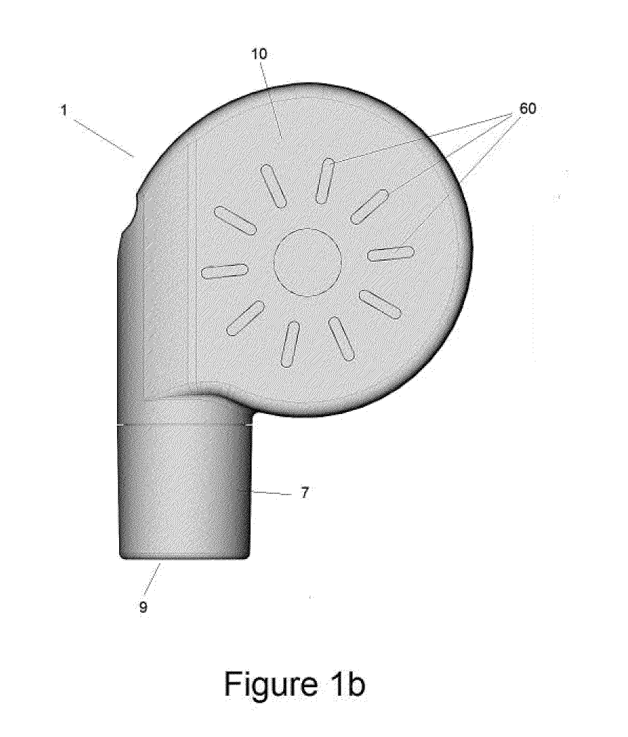

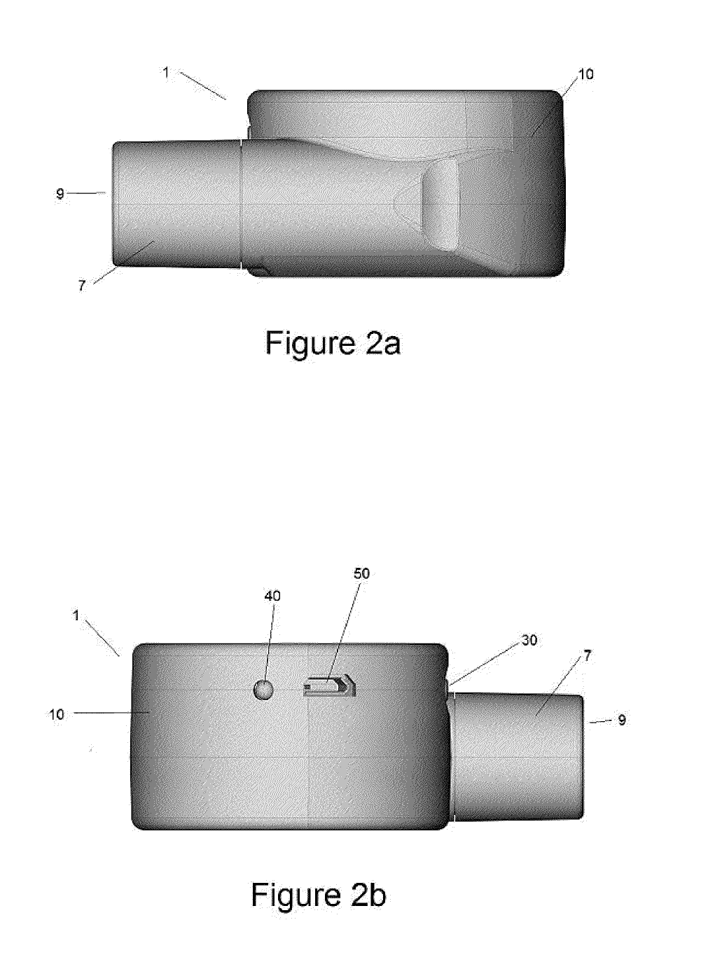

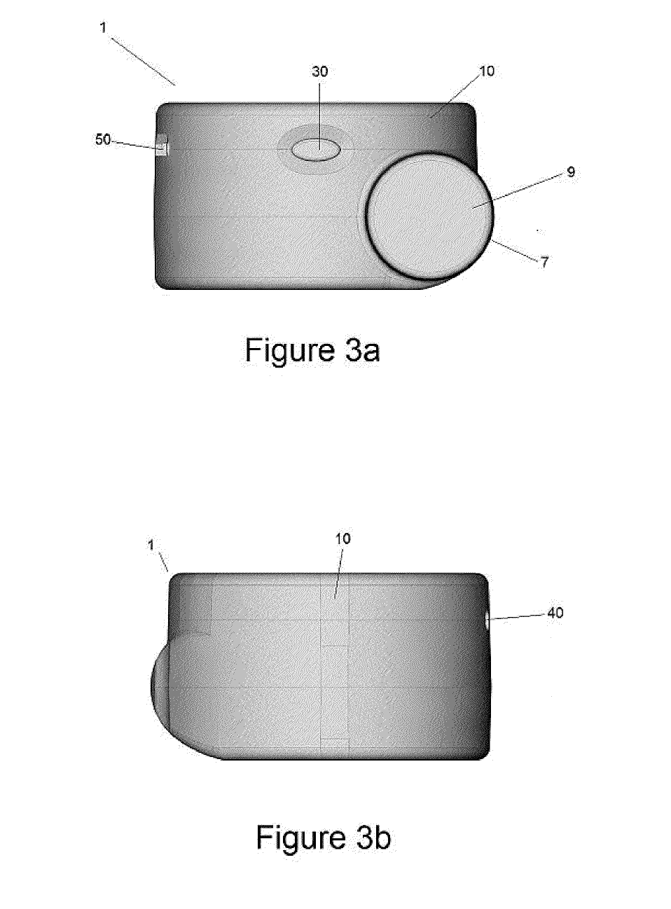

This device and system is an inhaler 1 that is an electronic device that uses air pressure to deliver, flavor, aroma and possibly active elements utilizing air pressure, airflow and room temperature volatilization or vaporization as shown in FIGS. 1a, 1b, 2a, 2b, 3a and 3b.

The inhaler 1 is an electronic device that uses air pressure to deliver, flavor, aroma and possibly active elements utilizing air pressure, airflow and room temperature volatilization or vaporization. Certain substances can have strong release of active elements and their aromatic elements at room temperature when exposed to air-pressure. The current invention is designed to maximize this when used.

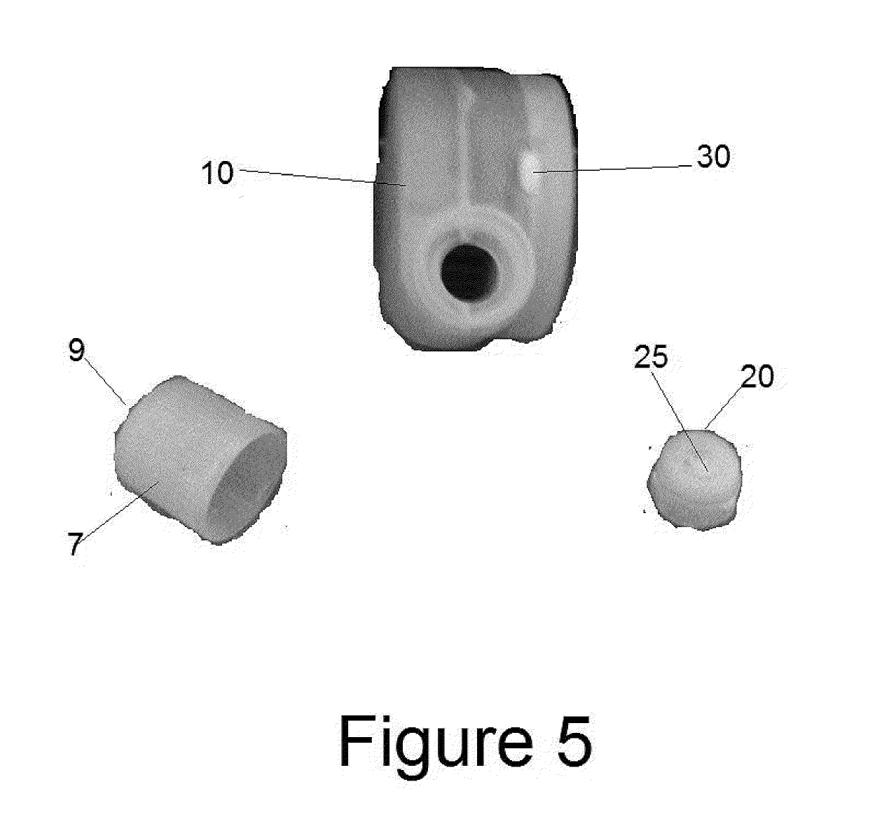

The inhaler 1 consists of a body 10 with a nozzle 7 at one end with a opening 9 allowing for the intake of air from the device 1. The inhaler 1 uses a ducted blower 10 to blow room temperature air 15 through a filter 20 embedded with a liquid, powder, paste or other source material 25. The user can then insert the nozzle 7 into the nose or mouth and inhale to enjoy the aroma and to receive the benefits of active elements as shown in FIG. 5. Examples of the active elements from the source materials 25 used include but are not limited to; Eucalyptus, Menthol, Caffeine, and/or Tobacco/Nicotine.

The device 1 will have a switch 30 that toggles it on and off. This switch 30 will be on the top of the body 10. The switch 30 is pressed down to on and release to off.

In the preferred embodiment, the device would have a super bright LED 40 inside the body 10 to light up the device 1 when in use. The device 1 can also have a USB port 50 to charge internal batteries or provide power.

The device 1 would be made of a use non glossy plastic for the body 10 and the nozzle 7. The device 1 should be flat plastic as shown in FIG. 1. A variety of colors can be used with the body 10 and the nozzle 7 being the same color or different colors. The body 10 would have rounded edges and air intake openings 60 and in the preferred embodiment be in the shape of a nine.

The filter 20 will be comprised of a small screen with a fine mesh. It is designed to be replaceable after a number of uses.

FIG. 5 shows the components of the device 1.

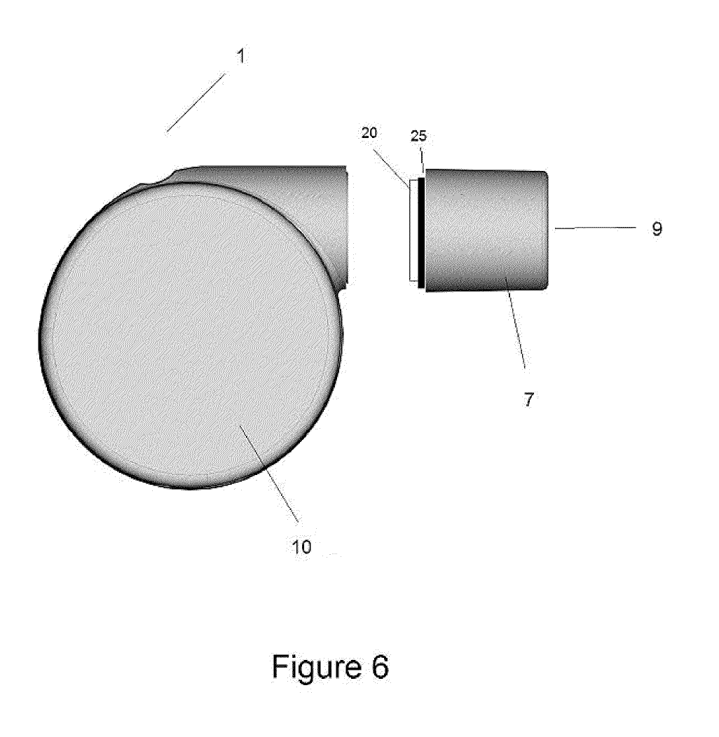

In an alternative embodiment, the filter 20 is attached to a disposable nozzle 7 as shown in FIG. 6, so both the nozzle 7 and the filter 20 can be throw away of a number of uses. This will increase the safety of the inhaler 1 while increasing the ease of replacing the filter.

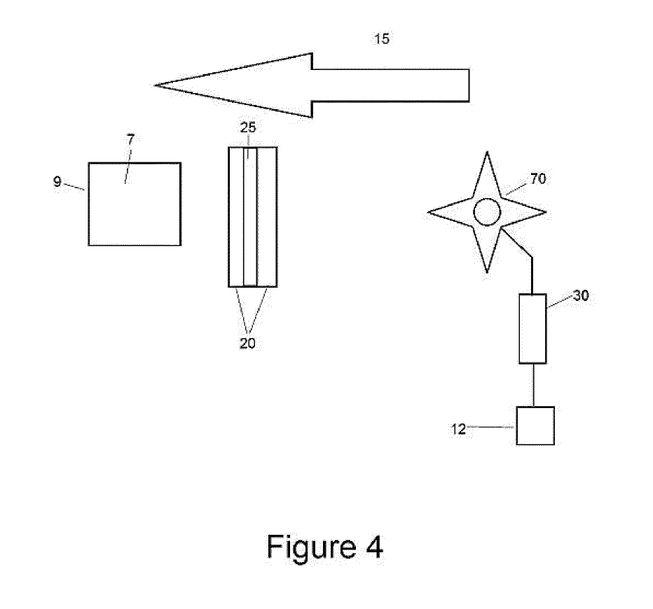

FIG. 4 shows the airflow 15 through the device 1. The air 15 is brought in air intake openings 60 through the filter 20 and through source material 25 driven by the ducted blower 70 to the nozzle 7. The ducted blower 70 consists of a fan like mechanism powered by a power source 12 such as a battery and is turned on and off by the switch 30. The ducted blower blows room temperature air 15 through a filter 20 that has been embedded with liquid, powder, paste or other source material 25.

The super bright LED 40 is also connected to the power source 12 and is turned on and off by the switch 30.

The filters 20 can be treated with the special menthol oils on them or other smells that the user may want. The nozzles may also be able to be preloaded with different source material 25.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur by those skilled in the art, it is not desired to limit the invention to the exact construction and operation shown and described, and accordingly, all suitable modifications and equivalents may be resorted to, falling within the scope of the invention.

I claim:

1. An inhaler device comprising: a body with an attached nozzle, within the body is a ducted blower with a filter containing a source material through to the nozzle.

2. A device according to claim 1 further comprising having the air being normal air temperature.

3. A device according to claim 1 further comprising being portable.

4. A device according to claim 1 further comprising the filter being comprised of a small screen with a fine mesh.

5. A device according to claim 1 further comprising the filter being replaceable.

6. A device according to claim 1 further comprising having air is brought in through a plurality of air intake openings then through the filter and through source material driven by the ducted blower to the nozzle.

7. A device according to claim 1 further comprising having the nozzle be disposable.

8. A device according to claim 1 further comprising having an LED.

9. A device according to claim 1 further comprising having the ducted blower 70 consist of a fan like mechanism.

10. A device according to claim 1 further comprising the ducted blower powered by a power source 12 and is turned on and off by a switch.

11. A device according to claim 1 further comprising being made of plastic.

12. A device according to claim 1 further comprising having the body 10 have rounded edges

13. A device according to claim 1 further comprising having the filter 20 embedded with one or more of a liquid, powder, or paste.

14. A device according to claim 1 further comprising having the filter 20 embedded with a menthol oils.

15. A device according to claim 1 further comprising having active elements from the source materials include one or more of the following; Eucalyptus, Menthol, Caffeine, Nicotine or Tobacco.

16. A device according to claim 1 further comprising being in the shape of a nine.

17. A device according to claim 1 further comprising having a USB port in the body.

18. A device according to claim 10 further comprising having a USB port in the body connected to the power source.

19. A portable inhaler device comprising: a body with an attached nozzle, within the body is a ducted blower with a filter containing a source material through to the nozzle where the filter being comprised of a small screen with a fine mesh and is replaceable where air is brought in through a plurality of air intake openings then through the filter and through source material driven by the ducted blower to the nozzle.

20. A device according to claim 19 further comprising having the nozzle be disposable.
